(19) United States
(12) Patent Application Publication
(10) Pub. No.: US 2003/0054334 A1
(43) Pub. Date: Mar. 20, 2003

(54) Medical and diagnostic use of a specific co-activator for human nuclear receptors

(76) Inventors: Maik Obendorf, Weimar (DE); Siemund Wolf, Jena (DE)

Correspondence Address: Striker Striker & Stenby, 103 East Neck Road, Huntington, NY, 11743, US

(21) Appl. No.: 10/169,365

(22) PCT Filed: Nov. 1, 2001
(86) PCT No.: PCT/EP01/12668

(30) Foreign Application Priority Data
Nov. 3, 2000 (DE) 10054639.0

(51) Int. Cl. C12Q 1/00; C12Q 1/68; G01N 33/53; G01N 33/567; A61K 31/56

(52) U.S. Cl. 435/4; 435/6; 435/7.2; 514/169

(57) ABSTRACT

The invention relates to a cloned gene encoding a protein which co-activates hormonal receptors, in particular the androgen receptor, in humans and relates, in addition, to the use of the co-activator protein as an important constituent in clinical tests for diagnoses of human clinical conditions.

The present invention relates to a cloned gene encoding a protein which co-activates hormonal receptors, in particular the androgen receptor, in humans and relates, in addition, to the use of the co-activator protein as an important constituent in clinical tests for diagnoses of human clinical conditions.

The nuclear receptor (NR) superfamily, consisting of over 50 different proteins, is a group of related transcription factors that regulate target gene transcription in response to specific ligands. The family can be divided into several subfamilies on the characteristics such as dimerization status, nature of ligand, or structure of the DNA response element (Beato et al., 2000, Human Reproduct. Update, 6, 225-236). The characteristics of NRs are the common functional domain structure (named A to F), including a highly variable, only weakly conserved N-terminal region that contains a constitutive autonomous activation function (AF-1), a highly conserved DNA binding domain (DBD) responsible for recognition of specific DNA response elements, consisting of two zinc-finger-like motifs, a variable hinge domain, and a conserved multifunctional C-terminal ligand binding domain (LBD), containing a dimerization and a ligand-dependent transactivation function (AF-2) followed by the most C-terminal region with unknown function which is absent in receptors such as PPAR (peroxisome proliferator-activated receptor) and RXR (retinoid X receptor) (Mangelsdorf & Evans, 1995; Cell, 83, 841-850; Robyr et al., 2000, Mol. Endocrinol., 14, 329-347). It was shown for some NR (e.g. AR) that the N-terminal region is able to interact with the C-terminal region (Brinkmann et al., 1999, J. Steroid Biochem. and Mol. Biol., 69, 307-313). Steroid hormone receptors such as estrogen receptor (ER), progesterone receptor (PR), glucocorticoid receptor (GR), mineralocorticoid receptor (MR), and androgen receptor (AR) bind steroidal ligands derived from pregnenolone, including progestins, estrogens, glucocorticoids, mineralocorticoids, and androgens. Ligand binding activates the receptor and regulates the expression of corresponding target genes.

In addition, another class of proteins, called co-modulators, was identified as a class of substances serving as bridging molecules between the transcription initiation complex and NRs to activate (co-activator) or repress (co-repressor) gene transcription (McKenna et al., 1999, Endocr. Rev., 20, 321-347). A co-activator must be able to enhance receptor function and to interact directly with the activation domain of NRs in an agonist-dependent manner but not in the presence of an antagonist. It also should interact with the basal transcription machinery, and finally it should not enhance the basal transcriptional activity by its own. Most of the co-modulators interact with the AF-2 domain of NRs through one or several LXXLL motifs (NR boxes) but some co-modulators showing interaction with other regions of NRs were also described (Ding et al., 1998, Mol. Endocrinol, 12, 302-313). In addition, many identified co-modulators interact with different NRs in a similar way so that the level of specificity for each isolated co-modulator has to be tested. To identify new potential co-aclivators, a fetal brain cDNA library was screened using fragments of the androgen receptor (AR) as probes. This was achieved by using the yeast two-hybrid system.

With the help of the invention a specific co-modulator for the human AR has been isolated, with interaction between AR and RanBPM. The gene for the co-modulator (RanBPM) has been described as a 55 kD protein of 500 amino acid residues localized within the centrosome involved in microtubule nucleation GenBank: NM_{—}005493) (Nakamura et al, 1998, J. Cell. Biol., 143, 1041-1052).

By cloning and reproduction of the AR activator gene the present invention also permits new laboratory tests to be developed to test the androgen specificity of candidate therapeutic substances.

The invention is particularly useful for

the use of RanBPM as target for pharmaceutical intervention of nuclear receptor dependent hormone signals,

use of RanBPM as target for screening of drug substances which transmit chemical signals through interaction with nuclear receptors and

the selective pharmaceutical intervention of the RanBPM-steroid hormone receptor interaction.

Furthermore the present invention provides a method for testing the hormonal, in particular the androgenic of antiandrogenic, effect of chemical substances in vitro. For example, such test comprises the steps of:

a) transforming host cells with a genetic construction effective in the host cell to produce both human nuclear receptor protein and RanBPM protein;

b) exposing the transformed host cells to the chemical substance; and

c) measuring the level of transcriptional activity caused by the nuclear receptor in the presence of RanBPM.

Other objects, advantages, and features of the present invention will become apparent from the following specification.

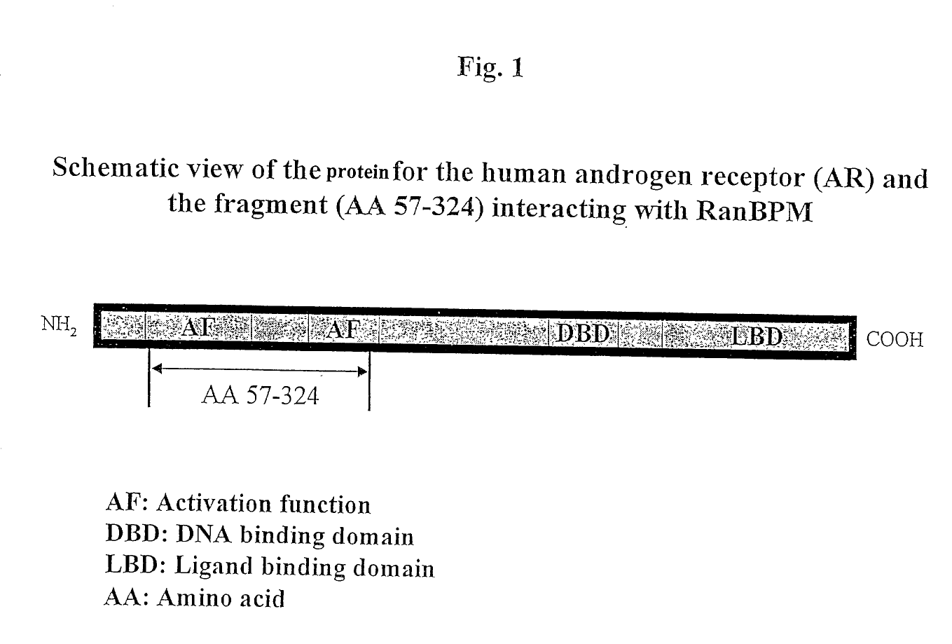

FIG. 1/1 is a schematic illustration of the AR depicting the AR domain between AA 57 and 324 which is able to interact with RanBPM.

The present invention is enabled by the isolation of a new regulatory protein in humans. This regulatory protein is the NR-associated protein, here designated RanBPM, which is a co-mediator, in particular co-activator, for the AR and other nuclear receptors, such as ER, PR, GR, MR, TR (thyroid hormone receptor), VDR (vitamin D receptor), PPAR, RAR (retinoid A receptor), and RXR. The RanBPM is a protein that can function as a specific co-mediator to enhance or repress the transcriptional effect of hormone binding to the nuclear receptor and also facilitates binding and activation of the nuclear receptor by molecules previously not thought to have hormonal effects.

In the following the present invention is illustrated by the AR as an example.

Using the yeast two hybrid system, a fetal brain cDNA library was screened with the human AR fragment encoding amino acid 57 to 324 as the probe (FIG. 1). One cDNA clone isolated during the screen was indistinguishable from a previously identified cDNA clone (RanBPM). (GenBank accession number NM_{—}005493; Nakamura et al, 1998, J. Cell. Biol., 143, 1041-1052).

It is specifically envisioned that RanBPM will have particular use as a constituent in a drug testing or screening protocol. It is a general practice in the evaluation of new clinical compounds for pharmaceutical use that the compounds be tested for possible hormonal activity. Androgenic or antiandrogenic side effects can be important in the administration of some pharmaceutical agents. Previously, methods used to test androgen activity measured the binding and activation ability of the androgen receptor transcriptional activity.

It is also anticipated that RanBPM will be used as an important clinical indicator for androgen related diseases. Significant androgen related diseases, such as prostate cancer, baldness, acne, hypogonadism, and androgen insensitive syndromes, such as Tfm syndrome, may be due to defects in the co-modulation mechanism between the AR and the RanBPM molecule. Accordingly, it becomes a reasonable possibility, to assay the relative ratios of AR and RanBPM in patients with such conditions. This is possible by raising antibodies to both RanBPM and to AR in performing quantitative methods to measure the relative quantity of the two molecules in a particular patient. Several methods exist for measuring such comparative ratios, including radio immunoassay, ELISA, immunostaining, RT-PCR or Western Blot. In addition, it would be possible to use the RanBPM cDNA so as to construct probes for PCR assay to identify mutations of the normal DNA sequence in particular patients, or to generate transcript for Northern Blot assay or DNA for in situ hybridization assays.

The theory for such measurements of relative ratios of AR to RanBPM is that androgen insensitivity-related disease may be due to an imbalance between AR and RanBPM prevalence in target cells. Too much RanBPM might over-sensitize the AR system, so as to be responsive to molecules not usually having an androgenic effect. Under-sensitivity due to absence or non-function of RanBPM may lead to androgen insensitivity at any levels. If too much RanBPM was found to be present in a particular patient, that would suggest the use of down regulation mechanism such as antisense or other similar mechanism, so as to clinically reduce the levels of RanBPM prevalent in a particular patient. This could also be achieved by molecules which can inhibit AR/RanBPM interaction. If a particular patient had too little RanBPM, then it would be possible o deliver RanBPM cDNA, protein, or DNA, into a patient by a variety of delivery mechanisms to increase levels of active RanBPM in the patient. It would also be possible to increase amount or activity of RanBPM by pharmaceutical drugs having low molecular weight, or by stimulation of its own synthesis using RanBPM-promoter specific proteins.

In addition to testing potential pharmaceutical uses, the RanBPM molecule would be useful also for testing non-pharmaceutical substances for potential androgenic/antiandrogenic activity. It is believed that many contaminants present within the environment at low levels have androgenic/antiandrogenic or estrogenic/antiestrogenic activity on various parts of the population.

To test samples for androgenic/antiandrogenic activity, genetic constructions including expression cassettes for both the androgen receptor and RanBPM would be transformed into eucaryotic host cells, such as a prostate cell line, neuronal cell line, and muscle cell line in vitro. Also an easily detectable and quantifiable detector gene would be transformed in the cells as well. A suitable detector (reporter) gene would be the gene encoding luciferase, or CAT (chloramphenicol acetyltransferase), or γ-galactosidase, or similar systems, and whose expression can be detected photometrically. The cells are then exposed to the effect of pharmaceutical agent or of the environmental sample material. Material with androgenic/antiandrogenic activity will then be recognized by increased or decreased reporter gene activity. It would also be possible to measure the influence of sample material on AR/RanBPM interaction by two-hybrid systems, immunoprecipitation, GST pull-down assays, gel retardation assays, FRET analysis, ABCD assays, etc.

### EXAMPLE

Using a fetal brain cDNA library and a human AR fragment encoding amino acid 57 to 324 as the probe (FIG. 1), a yeast two hybrid system screen was performed. According to the procedure of the manufacturer's protocol (Clontech) the number of screened clones was 3×10⁶, nearly the number of independent clones as indicated by the manufacturer (3.5×10⁶). 500 positives clones were selected and subjected to a γ-galactosidase assay where 200 of them were confirmed as lacZ positive clones. The inserts of these clones were amplified by PCR. After restriction fragment analyses and sequencing at least 17 different clones were found. One of them was a clone with a 2500 bp insert which encoded the entire ORF (open reading frame) of RanBPM (GenBank accession number NM_{—}005493). This clone was identified once during the library screen.

Here, we demonstrate that RanBPM binds to the human AR using a two hybrid system. This is the first demonstration of co-activator function of the RanBPM-protein for steroid receptors (e.g. AR). This interaction enables specific therapeutic intervention for several indications where hormones are involved in pathogenesis or therapy.

1. The use of RanBPM as target for pharmaceutical intervention of a nuclear receptor dependent hormone signalling system.

2. The use of RanBPM as target for screening of drug substances which transmit chemical signals through interaction with nuclear receptors.

3. The use according to claim 1 or 2, wherein the nuclear receptor is selected from the group consisting of the following receptors: androgen receptor (AR), estrogen receptor α (ERα), estrogen receptor β (ERβ), progesterone receptor A (PRA), progesterone receptor B (PR-B), glucocorticoid receptor (GR), and mineralocorticoid receptor (MR).

4. Pharmaceutical intervention (drug targeting) of the RanBPM/steroid hormone receptor interaction.

5. The pharmaceutical intervention according to claim 1 or 2, wherein the nuclear receptor is selected from the group consisting of the following receptors: androgen receptor (AR), estrogen receptor α (ERα), estrogen receptor β (ERβ), progesterone receptor A (PRA), progesterone receptor B (PR-B), glucocorticoid receptor (GR), and mineralocorticoid receptor (MR).

6. A method for testing the hormonal in particular the androgenic or antiandrogenic, effect of a chemical substance in vitro comprising the steps of
a) transforming host cells with a genetic construction effective in the host cells to produce, in future, both human nuclear receptor protein and RanBPM protein;
b) exposing the transformed host cells to the chemical substance; and
c) measuring the level of transcriptional activity caused by the nuclear receptor in the presence of RanBPM.

7. The method of claim 6, wherein the host cells are eucaryotic cells.

8. The method of claim 7, wherein the eucaryotic cells are prostate cells, neuronal cells or muscle cells.

9. The method of any of claims 6 to 8, wherein the chemical compound is a pharmaceutical.

10. The method of any of claims 6 to 8, wherein the chemical compound is contained in an environmental sample material.

11. The method of any of claims 6 to 10, wherein the nuclear receptor is selected from the group consisting of the following receptors: androgen receptor (AR), estrogen receptor α (ERα), estrogen receptor β (ERβ), progesterone receptor A (PRA), progesterone receptor B (PR-B), glucocorticoid receptor (GR), and mineralocorticoid receptor (MR).
